(19) United States
(12) Patent Application Publication
(10) Pub. No.: US 2011/0144398 A1
(43) Pub. Date: Jun. 16, 2011

(54) PROCESS FOR REGENERATING A RUTHENIUM-CONTAINING SUPPORTED HYDROGENATION CATALYST

(75) Inventors: Daniela Mirk, Speyer (DE); Christoph Schappert, Dudenhofen (DE); Uwe Stabel, Otterstadt (DE); Eberhardt Gaffron, Lampertheim (DE); Roman Prochazka, Mannheim (DE)

(73) Assignee: BASF SE, Ludwigshafen (DE)

(21) Appl. No.: 12/958,010

(22) Filed: Dec. 1, 2010

(30) Foreign Application Priority Data
Dec. 11, 2009 (EP) 09178939.6

(51) Int. Cl. C07C 13/18; B01J 38/06

(52) U.S. Cl. 585/269; 502/55

(57) ABSTRACT

Process for regenerating a ruthenium-containing supported hydrogenation catalyst. wherein the catalyst is treated with steam and then dried. Integrated process for hydrogenating benzene to cyclohexane in the presence of a ruthenium-containing supported catalyst, comprising, as well as the hydrogenation step. the catalyst regeneration steps.

The invention relates to a process for regenerating a ruthenium-containing supported hydrogenation catalyst.

Retention of catalyst activity over a maximum period is of great economic significance for industrial processes.

Typically, a decline in the catalytic activity is caused by various physical and chemical effects on the catalyst, for example by blocking of the catalytically active sites or by loss of catalytically active sites by thermal, mechanical or chemical processes. For example, catalyst deactivation or aging in general can be caused by sintering of the catalytically active sites, by loss of (noble) metal, by deposits or by poisoning of the active sites. The mechanisms of aging/deactivation are various.

Conventional wisdom is that the deactivated catalyst has to be removed from the reactor for regeneration. Thereafter, the reactor is idle, or operation is restarted after installation of another catalyst or switching to an already installed further catalyst. This leads to significant costs in each case. Patents U.S. Pat. No. 3,851,004 (Union Carbide Corp.) and U.S. Pat. No. 2,757,128 (Esso Res. & Eng. Comp.) disclose processes for hydrogenating olefins, inter alia, in hydrocarbon starting materials, and the regeneration of the catalysts by means of hydrogen.

DE 196 34 880 A1 (Intevep S.A.) discloses a process for simultaneous selective hydrogenation of diolefins and nitriles from a hydrocarbon starting material. In this process, the catalyst, once the diolefin hydrogenation activity thereof has fallen to less than 50% of the initial activity, is purged with an inert gas to remove traces of the hydrocarbon from the catalyst and to provide a purged catalyst, and purged with hydrogen in a subsequent regeneration step. This produces a regenerated catalyst, the diolefin hydrogenation activity of which again has at least 80% of the initial value.

JP 2008 238043 A relates to the regeneration of a reforming catalyst by means of steam at temperatures of ≧400° C.

WO 08/015103 A2 and WO 08/015170 A2 (both BASF AG) relate to processes for regenerating an Ru catalyst suitable for hydrogenation, comprising the purging of the catalyst with inert gas in a regeneration step until the original activity or a portion of the original activity is attained.

In the hydrogenation of benzene using the ruthenium catalysts described, a deactivation is likewise observed.

The object underlying the present invention was to provide a process for regenerating a ruthenium-containing hydrogenation catalyst, particularly a ruthenium catalyst used in hydrogenation of benzene. This process should be simple to implement in apparatus terms and be inexpensive to perform. More particularly, it should achieve multiple and complete regeneration of the catalyst.

Accordingly, a process has been found for regenerating a ruthenium-containing supported hydrogenation catalyst. which comprises treating the catalyst with steam and then drying it.

This reactivation firstly achieves higher conversions as a result of an increased catalyst activity; secondly, the process according to the invention significantly prolongs the catalyst service lives in productive operation.

The BET surface area (DIN ISO 9277) of the hydrogenation catalyst (fresh. before use thereof for hydrogenation) is preferably in the range from 100 to 250 m²/g, particularly in the range from 120 to 230 m²/g.

The process according to the invention is especially suitable for regenerating Ru catalysts which are described in patent applications EP 814 098 A2. WO 00/63142 A1 (EP 1 169 285 A1), WO 06/136541 A2 (DE 102 005 029 200 A) and WO 02/100537 A2 (all BASF AG) and are used in the processes disclosed there. These catalysts and processes are detailed below.

In the present document, the groups of the Periodic Table are designated in the CAS notation.

1.) EP 814 098 A2

The catalysts described hereinafter are referred to in the present application as “catalyst variant I”.

The active metals used may in principle be all metals of transition group VIII of the Periodic Table. The active metals used are preferably platinum, rhodium, palladium. cobalt, nickel or ruthenium, or a mixture of two or more thereof, ruthenium in particular being used as the active metal.

The terms “macropores” and “mesopores” are used in the context of the present invention as defined in Pure & Appl. Chem., Vol. 46, p. 79 (1976), specifically as pores whose diameter is above 50 nm (macropores) or whose diameter is between 2 nm and 50 nm (mesopores). “Micropores” are likewise defined according to the above literature and refer to pores with a diameter of <2 nm.

The active metal content is generally about 0.01 to about 30% by weight. preferably about 0.01 to about 5% by weight and especially about 0.1 to about 5% by weight, based in each case on the total weight of the catalyst used.

The metal surface area on catalyst variant I is, in total, preferably about 0.01 to about 10 m²/g, more preferably about 0.05 to about 5 m²/₉ and especially about 0.05 to about 3 m²/g of the catalyst.

The metal surface area is determined by means of the chemisorption process described by J. Lemaitre et al. in “Characterization of Heterogeneous Catalysts”, ed. Francis Delanney, Marcel Dekker, New York 1984, p. 310-324.

In catalyst variant I, the ratio of the surface areas of the active metal/metals and of the catalyst support is preferably less than about 0.05, where the lower limit is about 0.0005.

Catalyst variant I comprises a support material which is macroporous and has a mean pore diameter of at least about 50 nm, preferably at least about 100 nm, especially at least about 500 nm, and whose BET surface area is at most about 30 m²/g, preferably at most about 15 m²/g, more preferably at most about 10 m²/g, especially at most about 5 m²/g and more preferably at most about 3 m²/g. The mean pore diameter of the support is preferably about 100 nm to about 200 μm, more preferably about 500 nm to about 50 μm. The BET surface area of the support is preferably about 0.2 to about 15 m²/g, more preferably about 0.5 to about 10 m²/g, especially about 0.5 to about 5 m²/g and more preferably about 0.5 to about 3 m²/g.

The surface area of the support is determined by the BET method by N₂ adsorption, especially to DIN ISO 9277. The mean pore diameter and the, pore size distribution are determined by Hg porosimetry. especially to DIN 66133.

The pore size distribution of the support may preferably be approximately bimodal, the pore diameter distribution with maxima at about 600 nm and about 20 μm in the bimodal distribution constituting a specific embodiment.

Preference is further given to a support with a surface area of 1.75 m²/g, which has this bimodal distribution of the pore diameter. The pore volume of this preferred support is preferably about 0.53 ml/g.

Usable macroporous support materials are, for example, macropore-comprising activated carbon, silicon carbide, aluminium oxide, silicon dioxide, titanium dioxide, zirconium dioxide, magnesium oxide, zinc oxide or mixtures of two or more thereof, preference being given to using aluminum oxide and zirconium dioxide.

Corresponding catalyst supports and processes for preparation thereof are disclosed, for example, in the following documents:

Fundamentals of Industrial Catalytic Processes, R. J. Farrauto, C. H. Bartholomew, First Edition 1997, pages 16, 17, 57 to 62, 88 to 91, 110 to 111; Oberlander, R. K., 1984 Aluminas for Catalysts. in Applied Industrial Catalysis, D. E. Leach, Academic Press, Vol. 3, chapter 4: U.S. Pat. No. 3,245,919; WO 93/04774 A; EP 0 243 894 A; Ullmann's Encyclopedia of Industrial Chemistry, 5th Ed., Vol. A1, p. 588 to 590; VCH 1985.

2.) WO 00/63142 A1 (EP 1 169 285 A1)

The catalysts described hereinafter are referred to in the present application as “catalyst variant II”. There exist various subvariants of this variant II.

Subvariant 1

This catalyst corresponds to that described above under EP 0 814 098 A2.

In addition, the subvariant 1 a usable in accordance with the invention is described, which constitutes a preferred embodiment of subvariant 1. The usable support materials are those which are macroporous and have a mean pore diameter of at least 0.1 μm, preferably at least 0.5 μm, and a surface area of at most 15 m²/g, preferably at most 10 m²/g, more preferably at most 5 m²/g, especially at most 3 m²/g. The mean pore diameter of the support used there is preferably within a range from 0.1 to 200 μm, especially from 0.5 to 50 μm. The surface area of the support is preferably 0.2 to 15 m⁷/g, more preferably 0.5 to 10 m²/g, particularly 0.5 to 5 m²/g, especially 0.5 to 3 m²/g of the support. This catalyst too, with regard to the pore diameter distribution, has the bimodality already described above with the analogous distributions and the correspondingly preferred pore volumes.

Subvariant 2

Subvariant 2 comprises one or more metals of transition group VIII of the Periodic Table as active component(s) on a support, as defined herein. Preference is given to using ruthenium as the active component.

The metal surface area on the catalyst is, in total, preferably 0.01 to 10 m²/g, more preferably 0.05 to 5 m²/g and further preferably 0.05 to 3 m²/g of the catalyst. The metal surface area was measured by the chemisorption process as described in J. Lemaitre et al., “Characterization of Heterogeneous Catalysts”, ed. Francis Delanney, Marcel Dekker, New York (1984), p. 310-324.

In subvariant 2, the ratio of the surface areas of the at least one active metal and of the catalyst support is less than about 0.3, preferably less than about 0.1 and especially about 0.05 or less, the lower limit being about 0.0005.

The support materials usable in subvariant 2 possess macropores and mesopores.

The usable supports have a pore distribution, according to which about 5 to about 50%, preferably about 10 to about 45%, more preferably about 10 to about 30% and especially about 15 to about 25% of the pore volume is formed by macropores with pore diameters in the range from about 50 nm to about 10 000 nm, and about 50 to about 95%, preferably about 55 to about 90%, more preferably about 70 to about 90% and especially about 75 to about 85% of the pore volume by mesopores with a pore diameter of about 2 to about 50 nm, where the sum of the proportions of the pore volumes adds up to 100% in each case.

The total pore volume of the supports used is about 0.05 to 1.5 cm³/g, preferably 0.1 to 1.2 cm³/g and especially about 0.3 to 1.0 cm³/g. The mean pore diameter of the supports used in accordance with the invention is about 5 to 20 nm, preferably about 8 to about 15 nm and especially about 9 to about 12 nm.

The surface area of the support is preferably about 50 to about 500 m²/g, more preferably about 200 to about 350 m²/g and especially about 250 to about 300 m²/g of the support.

The surface area of the support is determined by the BET method by N₂ adsorption. especially to DIN ISO 9277. The mean pore diameter and the size distribution are determined by Hg porosimetry, especially to DIN 66133.

Even though it is possible in principle to use all support materials known in catalyst preparation, i.e. which have the above-defined pore size distribution, preference is given to using activated carbon, silicon carbide, aluminum oxide, silicon dioxide, titanium dioxide, zirconium dioxide, magnesium oxide, zinc oxide or mixtures thereof, more preferably aluminum oxide and zirconium dioxide.

3.) WO 06/136541 A2 (DE 102 005 029 200 A)

The catalysts described hereinafter are referred to in the present application as “catalyst variant III” or else “eggshell catalyst”.

The subject is an eggshell catalyst comprising, as an active metal, ruthenium alone or together with at least one further metal of transition groups IB, VIIB or VIII of the Periodic Table of the Elements (CAS notation), applied to a support comprising silicon dioxide as the support material.

In this eggshell catalyst, the amount of the active metal is <1% by weight, preferably 0.1 to 0.5% by weight, more preferably 0.25 to 0.35% by weight, based on the total weight of the catalyst, and at least 60% by weight, more preferably 80% by weight, of the active metal, based on the total amount of the active metal, is present in the shell of the catalyst down to a penetration depth of 200 μm. The aforementioned data are determined by means of SEM (scanning electron microscopy) EPMA (electron probe microanalysis)—EDXS (energy dispersive X-ray spectroscopy), and constitute averaged values. Further information with regard to the aforementioned analysis methods and techniques are disclosed, for example, in “Spectroscopy in Catalysis” by J. W. Niemantsverdriet, VCH, 1995.

The eggshell catalyst is notable in that the predominant amount of the active metal is present in the shell down to a penetration depth of 200 μm, i.e. close to the surface of the eggshell catalyst. In contrast, only a very small amount, if any, of the active metal is present in the interior (core) of the catalyst. This catalyst variant III has—in spite of the small amount of active metal—a very high activity in the hydrogenation of organic compounds which comprise hydrogenatable groups, especially in the hydrogenation of carbocyclic aromatic groups, coupled with very good selectivities. More particularly, the activity of catalyst variant III does not decrease over a long hydrogenation period.

Very particular preference is given to an eggshell catalyst in which no active metal can be detected in the interior of the catalyst, i.e. active metal is present only in the outermost shell, for example in a zone down to a penetration depth of 100 to 200 μm.

In a further particularly preferred embodiment, the eggshell catalyst is notable in that active metal particles can be detected in the (FEG)—TEM (Field Emission Gun—Transmission Electron Microscopy) with EDXS only in the outermost 200 μm, preferably 100 μm, most preferably 50 μm (penetration depth). Particles smaller than 1 nm cannot be detected.

As the active metal, ruthenium can be used alone or together with at least one further metal of transition groups IB, VIIB or VIII of the Periodic Table of the Elements (CAS notation). Further active metals suitable in addition to ruthenium are, for example, platinum, rhodium, palladium, iridium, cobalt or nickel, or a mixture of two or more thereof. Among the metals of transition groups IB and/or VIIB of the Periodic Table of the Elements which are likewise usable, for example, copper and/or rhenium are suitable. Preference is given to using ruthenium alone as the active metal, or together with platinum or iridium, in the eggshell catalyst; very particular preference is given to using ruthenium alone as the active metal.

The eggshell catalyst exhibits the aforementioned very high activity at a low loading with active metal, which is <1% by weight, based on the total weight of the catalyst. The amount of the active metal in the eggshell catalyst is preferably 0.1 to 0.5% by weight, more preferably 0.25 to 0.35% by weight. The penetration depth of the active metal into the support material depends on the loading of catalyst variant III with active metal. Even at a loading of catalyst variant III with 1% by weight or more, for example at a loading with 1.5% by weight, a significant amount of active metal is present in the interior of the catalyst, i.e. in a penetration depth of 300 to 1000 μm. which impairs the activity of the hydrogenation catalyst, especially the activity over a long hydrogenation period, especially in rapid reactions where a hydrogen deficiency can occur in the interior of the catalyst (core).

In the eggshell catalyst, at least 60% by weight of the active metal, based on the total amount of the active metal, is present in the shell of the catalyst down to a penetration depth of 200 μm. Preferably, at least 80% of the active metal in the eggshell catalyst. based on the total amount of the active metal, is present in the shell of the catalyst down to a penetration depth of 200 μm. Very particular preference is given to an eggshell catalyst in which no active metal can be detected in the interior of the catalyst. i.e. active metal is present only in the outermost shell, for example in a zone down to a penetration depth of 100 to 200 μm. In a further preferred embodiment, 60% by weight, preferably 80% by weight, based on the total amount of the active metal, is present in the shell of the catalyst down to a penetration depth of 150 μm. The aforementioned data are determined by means of SEM (scanning electron microscopy) EPMA (electron probe microanalysis)—EDXS (energy dispersive X-ray spectroscopy) and constitute averaged values. To determine the penetration depth of the active metal particles, several catalyst particles (e.g. 3, 4 or 5) are gently ground transverse to the extrudate axis (when the catalyst is in the form of extrudates). By means of linescans, the profiles of the active metal/Si concentration ratios are then detected. On each measurement line, several measurement points, for example 15 to 20. are measured at equal intervals; the measurement spot size is approximately 10 μm·10 μm. After integration of the amount of active metal over the depth. the frequency of the active metal in a zone can be determined.

Most preferably, the amount of the active metal, based on the concentration ratio of active metal to Si, at the surface of the eggshell catalyst is 2 to 25%, preferably 4 to 10%. more preferably 4 to 6%, determined by means of SEM EPMA—EDXS. The surface is analyzed by means of area analyses of areas of 800 μm×2000 μm and with an information depth of approximately 2 μm. The element composition is determined in % by weight (normalized to 100%). The mean concentration ratio (active metal/Si) is averaged over 10 measurement areas.

The surface of the eggshell catalyst is understood to mean the outer shell of the catalyst down to a penetration depth of approximately 2 μm. This penetration depth corresponds to the information depth in the aforementioned surface analysis.

Very particular preference is given to an eggshell catalyst in which the amount of the active metal, based on the weight ratio of active metal to Si (wt./wt. in %), at the surface of the eggshell catalyst is 4 to 6%, at a penetration depth of 50 μm 1.5 to 3%, and in the penetration depth range from 50 to 150 μm 0.5 to 2%, determined by means of SEM EPMA (EDXS). The values mentioned constitute averaged values.

In addition, the size of the active metal particles preferably decreases with increasing penetration depth, determined by means of (FEG)—TEM analysis.

The active metal is preferably present partly or fully in crystalline form in the eggshell catalyst. In preferred cases, ultrafine crystalline active metal can be detected in the shell of the eggshell catalyst by means of SAD (selected area diffraction) or XRD (X-ray diffraction).

The eggshell catalyst may additionally comprise alkaline earth metal ions (M²⁺), i.e. M=Be, Mg, Ca, Sr and/or Ba, especially Mg and/or Ca, very particularly Mg. The content of alkaline earth metal ion(s) (M²⁺) in the catalyst is preferably 0.01 to 1% by weight, especially 0.05 to 0.5% by weight, very particularly 0.1 to 0.25% by weight, based in each case on the weight of the silicon dioxide support material.

A significant constituent of catalyst variant III is the support material based on silicon dioxide, generally amorphous silicon dioxide. The term “amorphous” in this context is understood to mean that the proportion of crystalline silicon dioxide phases makes up less than 10% by weight of the support material. The support materials used to prepare the catalysts may, however, have superstructures which are formed by regular arrangement of pores in the support material.

Useful support materials are in principle amorphous silicon dioxide types which consist at least to an extent of 90% by weight of silicon dioxide, though the remaining 10% by weight, preferably not more than 5% by weight, of the support material may also be another oxidic material, e.g. MgO, CaO, TiO₂, ZrO₂, Fe₂O₃ and/or alkali metal oxide.

In a preferred embodiment, the support material is halogen-free, more particularly chlorine-free. i.e. the content of halogen in the support material is less than 500 ppm by weight, for example in the range from 0 to 400 ppm by weight. Preference is thus given to an eggshell catalyst which comprises less than 0.05% by weight of halide (determined by ion chromatography), based on the total weight of the catalyst.

Preference is given to support materials which have a specific surface area in the range from 30 to 700 m²/g, preferably 30 to 450 m²/g (BET surface area to DIN ISO 9277).

Suitable amorphous support materials based on silicon dioxide are familiar to the person skilled in the art and are commercially available (see, for example, O. W. Flörke, “Silica” in Ullmann's Encyclopedia of Industrial Chemistry 6th Edition on CD-ROM). They may either be of natural origin or may have been synthetically produced. Examples of suitable amorphous support materials based on silicon dioxide are silica gels, kieselguhr, fumed silicas and precipitated silicas. In a preferred embodiment of the invention, the catalysts have silica gels as support materials.

According to the configuration, the support material may have a different shape. When the process in which the eggshell catalysts are used is configured as a suspension process, the catalysts will typically be prepared using the support material in the form of a fine powder. The powder preferably has particle sizes in the range from 1 to 200 μm, especially 1 to 100 μm. In the case of use of the eggshell catalyst in fixed catalyst beds, it is customary to use shaped bodies of the support material, which are obtainable, for example, by extruding or tableting and which may have, for example, the form of spheres, tablets, cylinders, extrudates, rings or hollow cylinders, stars and the like. The dimensions of these shaped bodies vary typically within the range from 0.5 mm to 25 mm. Frequently, catalyst extrudates with extrudate diameters of 1.0 to 5 mm and extrudate lengths of 2 to 25 mm are used. With smaller extrudates, it is generally possible to achieve higher activities, but they often exhibit insufficient mechanical stability in the hydrogenation process. Very particular preference is therefore given to using extrudates with extrudate diameters in the range from 1.5 to 3 mm.

4.) WO 02/100537 A2

The catalysts described hereinafter are referred to in the present application as “catalyst variant IV”.

The ruthenium catalyst according to this catalyst variant IV is obtainable by:

i) treating a support material based on amorphous silicon dioxide once or more than once with a halogen-free aqueous solution of a low molecular weight ruthenium compound and then drying the treated support material at a temperature below 200° C., particularly ≦150° C.

ii) reducing the solid obtained in i) with hydrogen at a temperature in the range from 100 to 350° C., particularly in the range from 150 to 320° C., step ii) being performed directly after step i).

The support based on amorphous silicon dioxide preferably has a BET surface area (DIN ISO 9277) in the range from 50 to 700 m²/g.

More particularly, the ruthenium catalyst comprises ruthenium in an amount of 0.2 to 10% by weight, particularly 0.2 to 7% by weight, more particularly 0.4 to 5% by weight, based in each case on the weight of the support.

Preference is given to an embodiment of the ruthenium catalyst in which the support material based on silicon dioxide consists to an extent of at least 90% by weight of silicon dioxide and comprises less than 1% by weight of aluminum oxide. calculated as Al₂O₃.

The ruthenium compound used in step i) is preferably selected from ruthenium (III) nitrosylnitrate, ruthenium (III) acetate, sodium ruthenate (IV) and potassium ruthenate (IV).

The solid which is obtained from i) and used for reduction in ii) preferably has a water content of less than 5% by weight, preferably less than 2% by weight, based in each case on the total weight of the solid.

More particularly, the drying in step i) is effected with movement of the treated support material.

Particular preference is given to an embodiment of the ruthenium catalyst comprising less than 0.05% by weight of halogen, particularly less than 0.01% by weight of halogen, based in each case on the total weight of the catalyst, and consisting of:
a support material based on amorphous silicon dioxide and
elemental ruthenium present on the support in atomically dispersed form and/or in the form of ruthenium particles,
   the catalyst having essentially no ruthenium particles and/or agglomerates with diameters above 10 nm.

The hydrogenation catalyst, particularly one of the above-described catalysts (catalyst variants I, II, III and IV and subvariants mentioned), of the process according to the invention is preferably used for ring hydrogenation of an aromatic organic compound. It is used especially for hydrogenation of a carbocyclic aromatic group to the corresponding carbocyclic aliphatic group. Particular preference is given to fully hydrogenating the aromatic group.

Particular preference is given to using the hydrogenation catalyst to hydrogenate benzene to cyclohexane.

Full hydrogenation is understood to mean a conversion of reactant (e.g. benzene) of generally >98%. preferably >99%, more preferably >99.5%, even more preferably >99.9%, particularly >99.99% and especially >99.995%.

Especially in the case of use of the above-described catalyst variants I, II, III and IV (including subvariants mentioned) for hydrogenation of benzene to cyclohexane, the typical cyclohexane specifications which require a residual benzene content of <100 ppm by weight (which corresponds to a benzene conversion of >99.99%) are thus complied with. As mentioned, the benzene conversion in a hydrogenation of benzene, especially with the abovementioned eggshell catalyst, is preferably >99.995%.

The present application therefore further provides an integrated process for hydrogenating benzene to cyclohexane in the presence of an Ru-containing supported catalyst, which, as well as the hydrogenation step. comprises the inventive regeneration steps.

The hydrogenation step can be performed in the liquid phase or in the gas phase. Preference is given to performing the hydrogenation step in the liquid phase.

The hydrogenation step can be performed in the absence of a solvent or diluent or in the presence of a solvent or diluent, i.e. it is not necessary to perform the hydrogenation in solution.

The solvent or diluent used may be any suitable solvent or diluent. Useful solvents or diluents are in principle those which are capable of dissolving the organic compound to be hydrogenated very substantially completely or mix completely with the latter and are inert under the hydrogenation conditions, i.e. are not hydrogenated.

Examples of suitable solvents are cyclic and acyclic ethers, for example tetrahydrofuran, dioxane. methyl tert-butyl ether, dimethoxyethane, dimethoxypropane, dimethyldiethylene glycol, aliphatic alcohols such as methanol, ethanol, n- or isopropanol, n-, 2-, iso- or tert-butanol, carboxylic esters such as methyl acetate, ethyl acetate. propyl acetate or butyl acetate, and aliphatic ether alcohols such as methoxypropanol, and cycloaliphatic compounds such as cyclohexane, methylcyclohexane and dimethylcyclohexane.

The amount of the solvent or diluent used is not restricted in any particular way and can be selected freely as required, although preference is given to those amounts which lead to a 3 to 70% by weight solution of the organic compound intended for hydrogenation. The use of a diluent is advantageous in order to prevent excessive exothermicity in the hydrogenation process. Excessive exothermicity can lead to deactivation of the catalyst and is therefore undesirable. Careful temperature control is therefore advisable in the hydrogenation step. Suitable hydrogenation temperatures are specified below.

In the case of use of a solvent, particular preference is given to using the product formed in the hydrogenation, i.e. cyclohexane, as the solvent, optionally in addition to other solvents or diluents. In each case, a portion of the cyclohexane formed in the process can be added to the benzene which is still to be hydrogenated.

Based on the weight of the benzene intended for hydrogenation, preference is given to adding 1 to 30 times, more preferably 5 to 20 times, especially 5 to 10 times, the amount of the cyclohexane product as a solvent or diluent.

The actual hydrogenation is typically effected by contacting the organic compound as the liquid phase or gas phase, preferably as the liquid phase. with the catalyst in the presence of hydrogen. The liquid phase can be passed through a catalyst suspension (suspension mode) or a fixed catalyst bed (fixed bed mode).

The hydrogenation can be configured either continuously or batchwise, preference being given to the continuous process procedure. Preference is given to performing the process in trickle reactors or in flooded mode by the fixed bed method. The hydrogen can be passed over the catalyst either in cocurrent with the solution of the reactant to be hydrogenated or in countercurrent.

Suitable apparatus for performing a hydrogenation over the fluidized catalyst bed and over the fixed catalyst bed are known from the prior art. for example from Ullmanns Enzyklopädie der Technischen Chemie, 4th edition, volume 13, p. 135 ff., and from P. N. Rylander, “Hydrogenation and Dehydrogenation” in Ullmann's Encyclopedia of Industrial Chemistry, 5th ed. on CD-ROM.

The hydrogenation can be performed either at standard hydrogen pressure or at elevated hydrogen pressure for example at an absolute hydrogen pressure of at least 1.1 bar, preferably at least 2 bar. In general, the absolute hydrogen pressure will not exceed a value of 325 bar and preferably 300 bar. The absolute hydrogen pressure is more preferably in the range from 1.1 to 300 bar. most preferably in the range from 5 to 40 bar. The hydrogenation of benzene is effected. for example, at a hydrogen pressure of generally ≦50 bar, preferably 10 bar to 45 bar, more preferably 15 to 40 bar.

The reaction temperatures in the hydrogenation step are generally at least 30° C. and will frequently not exceed a value of 250° C. The hydrogenation step is preferably performed at temperatures in the range from 50 to 200° C., more preferably 70 to 180° C. and even more preferably in the range from 80 to 160° C. The hydrogenation of benzene is most preferably performed at temperatures in the range from 75 to 170° C., especially 80 to 160° C.

Useful reaction gases include, as well as hydrogen, also hydrogenous gases which do not comprise any catalyst poisons such as carbon monoxide or sulfur-containing gases such as H₂S or COS, for example mixtures of hydrogen with inert gases such as nitrogen, or reformer offgases which typically still comprise volatile hydrocarbons. Preference is given to using pure hydrogen (purity ≧99.9% by volume, particularly ≧99.95% by volume, especially ≧99.99% by volume).

Owing to the high catalyst activity, comparatively small amounts of catalyst are required, based on the reactant used. Thus, in the batchwise suspension mode, preferably less than 5 mol%, for example 0.2 mol% to 2 mol%, of active metal. based on 1 mol of reactant, will be used. In a continuous configuration of the hydrogenation process, the reactant to be hydrogenated will typically be conducted over the catalyst with a space velocity of 0.05 to 3 kg/(I(catalyst)·h), especially 0.15 to 2 kg/(I(catalyst)·h).

Very particularly preferred features of the hydrogenation step

The aromatic hydrogenation comprising the inventive regeneration steps is effected generally at a temperature of 75 to 170° C., preferably 80 to 160° C. The pressure is generally ≦50 bar, preferably 10 to 45 bar, more preferably 15 to 40 bar, most preferably 18 to 38 bar.

Preference is given to hydrogenating benzene to cyclohexane at an absolute pressure of about 20 bar.

The benzene used in the hydrogenation step has, in a preferred embodiment of the process according to the invention, a sulfur content of generally ≦2 mg/kg. preferably ≦1 mg/kg, more preferably ≦0.5 mg/kg, even more preferably ≦0.2 mg/kg and especially ≦0.1 mg/kg. A sulfur content of ≦0.1 mg/kg means that no sulfur is detected in the benzene with the test method specified below.

Test method: Determination according to Wickbold (DIN EN 41), followed by ion chromatography.

The hydrogenation can generally be performed in suspension or fixed bed mode, preference being given to performance in fixed bed mode. Particular preference is given to performing the hydrogenation process with liquid circulation, in which case the heat of hydrogenation can be removed by means of a heat exchanger and utilized. The feed/circulation ratio in the case of performance of the hydrogenation process with liquid circulation is generally 1:5 to 1:40, preferably 1:10 to 1:30.

In order to achieve full conversion, a postreaction of the hydrogenation discharge can be effected. For this purpose, the hydrogenation discharge, after the hydrogenation process, can be passed through a downstream reactor in straight pass in the gas phase or in the liquid phase. The reactor can be operated in trickle mode or in flooded mode in the case of liquid phase hydrogenation. The reactor is filled with the inventive catalyst or with another catalyst known to those skilled in the art.

The hydrogen used in the hydrogenation step preferably does not comprise any harmful catalyst poisons, for example CO. For example, reformer gases can be used. Preference is given to using pure hydrogen as the hydrogenation gas.

The inventive regeneration steps

In hydrogenation processes in which, for example, the Ru catalysts detailed above are used, deactivation is observed after a certain catalyst service life. Such a deactivated ruthenium catalyst can, in accordance with the invention, be returned to the state of original activity by treatment with steam and subsequent drying. The activity can be restored particularly up to >90%, preferably >95%, more preferably >98%, especially >99%, most preferably >99.5%, of the starting value (i.e. of the activity of the fresh catalyst before the hydrogenation step, i.e. before the use thereof in the hydrogenation).

The treatment with steam is performed preferably at a temperature in the range from 100 to 200° C., particularly 105 to 150° C., very particularly 110 to 130° C.

The treatment with steam is preferably performed at an absolute pressure in the range from 0.5 to 10 bar, particularly 0.8 to 8 bar. very particularly 0.9 to 4 bar.

The treatment with steam is preferably performed over a period in the range from 10 to 200 hours, particularly 20 to 150 hours, very particularly 50 to 100 hours.

The treatment with steam is preferably performed continuously with a flow rate of 100 to 400 kg (steam), preferably 150 to 350 kg, very particularly 200 to 300 kg. per square meter (cross-sectional catalyst area of the catalyst bed) and per hour [kg/(m²·h)].

The drying is preferably performed directly after the treatment with steam.

The drying is preferably performed at a temperature in the range from 10 to 350° C., particularly 50 to 250° C., very particularly 70 to 180° C., more particularly 80 to 130° C.

The drying is preferably performed at an absolute pressure in the range from 0.5 to 5 bar, particularly 0.8 to 2 bar. very particularly 0.9 to 1.5 bar.

The drying is preferably performed over a period in the range from 10 to 50 hours, particularly 10 to 20 hours.

The drying is preferably effected by purging with a gas or gas mixture. For example, the calculated drying time of the catalyst bed of an industrial scale cyclohexane production plant with an assumed moisture content of 2 or 5% by weight is approximately 18 or 30 hours. The purging can be performed in the process according to the invention either in the downward direction (downflow) or in the upward direction (upflow).

“Purging” means that the catalyst is contacted with the gas or gas mixture. For this purpose, the gas or gas mixture is normally passed over the catalyst by suitable constructive measures known to those skilled in the art.

The gas is preferably selected from nitrogen, oxygen, carbon dioxide, helium, argon, neon and mixtures thereof. Air is most preferred.

In a particular embodiment of the invention, the process according to the invention for regeneration is performed without deinstallation of the catalyst in the same reactor in which the hydrogenation took place. In a particularly advantageous manner, the drying of the catalyst, especially by purging with a gas or gas mixture, is performed in the reactor at temperatures and pressures which correspond to or are similar to the hydrogenation reaction, which results in only a very brief interruption to the reaction process.

The purging with gas or gas mixture is preferably performed continuously with a volume flow rate in the range from 20 to 200 standard liters per hour and per liter of catalyst (bed volume) [I (STP)/(lcat.·h)], preferably 50 to 200 I (STP)/(lcat.·h).

The present invention further provides an integrated process for hydrogenating benzene in the presence of a ruthenium catalyst, comprising the inventive catalyst regeneration steps. and comprising the following steps:

(a) providing benzene and a ruthenium catalyst,

(b) hydrogenating the benzene by contacting with hydrogen in the presence of the ruthenium-containing catalyst until the catalyst has a reduced hydrogenation activity,

(c) the catalyst regeneration steps, (

d) optionally repeating steps (a) to (c).

All pressure figures relate to the absolute pressure. BET surface areas always according to DIN ISO 9277:1995.

### EXAMPLES

### Example 1

Preparation of an Ru/Al₂O₃ Catalyst

An Ru/Al₂O₃ catalyst comprising 0.5% by weight of ruthenium was prepared analogously to the manner described in EP 814 098 A2 (BASF AG). page 14 lines 20 to 29.

### Example 2

Preparation of an Ru/SiO₂ Catalyst

An Ru/SiO₂ catalyst comprising 0.32% by weight of ruthenium was prepared as described in WO 2006/136541 A2 (BASF AG), page 30 line 31 to page 33 line 18 (“catalyst G” therein).

### Example 3

Deinstallation of the spent Ru/Al₂O₃ catalyst according to example 1, regeneration by steam treatment and drying

After the complete deactivation thereof in the hydrogenation of benzene, the catalyst prepared according to example 1 was deinstalled. To this end, the reactor was first emptied and the catalyst bed which was still at approx. 110° C. was purged with 2.5 t/h of steam. Once the steam was free of organic carbon, the bed, which was at first at approx. 110° C. was purged first with nitrogen and then with air, which cooled the bed.

### Example 4

Deinstallation of the spent Ru/SiO₂ catalyst according to example 2, regeneration by steam treatment and drying

The Ru/SiO₂ catalyst which was prepared according to example 2 was deinstalled after the complete deactivation thereof in the hydrogenation of benzene analogously to the procedure described in example 3.

### Example 5

Activity Test of the Deinstalled Ru/Al₂O₃ Catalyst

An activity test was performed with the Ru/Al₂O₃ catalyst treated by the procedure described in example 3:

A continuously operated plant with a 90 ml tubular reactor was charged with 2 samples of the spent catalyst (each 90 ml: 75 g of a sample from the lower quarter of the hydrogenation reactor of a cyclohexane plant, 72 g of a sample from the upper quarter of the hydrogenation reactor of a cyclohexane plant). The reactor was operated in trickle mode with circulation. 60.6 ml/h of benzene and 58 I (STP)/h of hydrogen were conducted through the reactor at inlet temperature 75° C. and outlet temperature 125° C., a pressure of 30 bar and circulation rate 1.5 kg/h (l (STP)=standard liters=volume converted to standard conditions (20° C., 1 bar absolute)). The gas chromatography analysis of the reaction discharge showed that the benzene had been converted to an extent of >99.5%. Compared to the unused original catalyst sample (see example 6), the conversion at run time 50 h was significantly better.

### Example 6

Comparative Example with Unused (fresh) Ru/Al₂O₃ Catalyst

Analogously to example 5, a test was performed with fresh catalyst (prepared according to example 1):

A continuously operated plant with a 90 ml tubular reactor was charged with a sample of the catalyst set aside (90 ml. 62 g of a sample which had been set aside). The reactor was operated in trickle mode with circulation. 60.6 ml/h of benzene and 58 l (STP)/h of hydrogen were conducted through the reactor at inlet temperature 75° C. and outlet temperature 125° C., a pressure of 30 bar and circulation rate 1.5 kg/h. The gas chromatography analysis of the reaction discharge showed that the benzene had been converted to an extent of >99.5%.

### Example 7

Activity Test of the Deinstalled Ru/SiO₂ Catalyst

An activity test was performed with the Ru/SiO₂ catalyst treated by the procedure described in example 4:

A 300 ml pressure reactor was initially charged with 2.5 g of the spent catalyst in a catalyst insert basket and admixed with 100 g of 5% by weight benzene in cyclohexane. The hydrogenation was performed with pure hydrogen at a constant pressure of 32 bar and a temperature of 100° C. Hydrogenation was effected for 4 h.

The reactor was subsequently decompressed. The gas chromatography analysis of the reaction discharge showed that the benzene had been converted to an extent of >99.5%.

### Example 8

Comparative Example with Unused (Fresh) Ru/SiO₂ Catalyst

Analogously to example 7, a test was performed with fresh catalyst (prepared according to example 2):

A 300 ml pressure reactor was initially charged with 2.5 g of the unused (fresh) catalyst in a catalyst insert basket and admixed with 100 g of 5% by weight benzene in cyclohexane. The hydrogenation was performed with pure hydrogen at a constant pressure of 32 bar and a temperature of 100° C. Hydrogenation was effected for 4 h. The reactor was subsequently decompressed. The gas chromatography analysis of the reaction discharge showed that the benzene had been converted to an extent of >99.5%.

1. A process for regenerating a ruthenium-containing supported hydrogenation catalyst, which comprises treating the catalyst with steam and then drying the catalyst.

2. The process according to claim 1, wherein the treatment with steam is performed at a temperature in the range from 100 to 200° C.

3. The process according to claim 1, wherein the treatment with steam is performed at an absolute pressure in the range from 1 to 10 bar.

4. The process according to claim 1, wherein the treatment with steam is performed over a period in the range from 10 to 100 hours.

5. The process according to claim 1, wherein the treatment with steam is performed continuously with a flow rate of 100 to 400 kg per square meter and per hour [kg/(m²·h)].

6. The process according to claim 1, wherein the drying is performed at a temperature in the range from 10 to 350° C.

7. The process according to claim 1, wherein the drying is performed at an absolute pressure in the range from 0.5 to 5 bar.

8. The process according to claim 1, wherein the drying is performed over a period in the range from 10 to 50 hours.

9. The process according to claim 1, wherein the drying is effected by purging with a gas or gas mixture.

10. The process according to claim 1, wherein the gas is selected from nitrogen, oxygen, carbon dioxide, helium, argon, neon and mixtures thereof.

11. The process according to claim 9, wherein the gas mixture is air.

12. The process according to claim 9, wherein the purging with gas or gas mixture is performed continuously with a volume flow rate in the range from 20 to 200 standard liters per hour and per liter of catalyst [l (STP)/(lcat.·h)].

13. The process according to claim 1, wherein the ruthenium-containing catalyst is an aluminum oxide- and/or silicon dioxide-supported catalyst.

14. The process according to claim 1, wherein the ruthenium-containing catalyst is one of the following catalysts:
a) catalyst comprising, as an active metal, ruthenium alone or together with at least one metal of transition group 1. VII or VIII of the Periodic Table (CAS notation) in an amount of 0.01 to 30% by weight, based on the total weight of the catalyst, applied to a support, and where 10 to 50% of the pore volume of the support is formed by macropores with a pore diameter in the range from 50 nm to 10000 nm, and 50 to 90% of the pore volume of the support by mesopores with a pore diameter in the range from 2 to 50 nm, where the sum of the pore volumes adds up to 100%,
b) eggshell catalyst comprising, as an active metal, ruthenium alone or together with at least one further metal of transition groups IB, VIIB or VIII of the Periodic Table of the Elements (CAS notation), applied to a support comprising silicon dioxide as the support material, where the amount of the active metal is ≦1% by weight, based on the total weight of the catalyst, and at least 60% by weight of the active metal is present in the shell of the catalyst down to a penetration depth of 200 μm, determined by means of SEM—EPMA (EDXS).

15. The process according to claim 14, wherein at least one metal of transition group I, VII or VIII of the Periodic Table in catalyst a) is platinum, copper, rhenium, cobalt, nickel or a mixture of two or more. thereof.

16. The process according to claim 14, wherein the support in catalyst a) is activated carbon, silicon carbide, aluminum oxide, silicon dioxide, titanium dioxide, zirconium dioxide, magnesium oxide, zinc oxide or a mixture of two or more thereof.

17. The process according to claim 1, wherein the BET surface area of the catalyst (DIN ISO 9277) is in the range from 100 to 250 m²/g.

18. The process according to claim 1, wherein the hydrogenation catalyst is used for ring hydrogenation of an aromatic Organic compound.

19. The process according to claim 1, wherein the hydrogenation catalyst is used to hydrogenate benzene to cyclohexane.

20. An integrated process for hydrogenating benzene to cyclohexane in the presence of a ruthenium-containing supported catalyst, comprising, as well as the hydrogenation step, the catalyst regeneration steps as described in claim 1.

21. The process according to claim 20, comprising the following steps:
a) providing benzene and a ruthenium-containing catalyst,
b) hydrogenating the benzene by contacting with hydrogen in the presence of the ruthenium-containing catalyst until the catalyst has a reduced hydrogenation activity,
c) the catalyst regeneration steps,
d) optionally repeating steps a) to c).

22. The process according to claim 1, wherein the regeneration steps establish a catalyst activity of >90% of the activity before the hydrogenation step.
